(19) United States
(12) Patent Application Publication
(10) Pub. No.: US 2024/0277851 A1
(43) Pub. Date: Aug. 22, 2024

(54) siRNA DELIVERY VECTOR

(71) Applicant: COUNCIL OF SCIENTIFIC & INDUSTRIAL RESEARCH, New Delhi (IN)

(72) Inventors: Srigiridhar KOTAMRAJU, Hyderabad, Telangana (IN); Surendar Reddy BATHULA, Hyderabad, Telangana (IN); Altab SHAIKH, Hyderabad, Telangana (IN); Praveen Kumar NEELI, Hyderabad, Telangana (IN); Panangipalli SRAVYA, Hyderabad, Telangana (IN); Rajkumar BANERJEE, Hyderabad, Telangana (IN); Rajamannar THENNATI, Vadodara, Gujurat (IN); Singuru GAJALAKSHMI, Hyderabad, Telangana (IN)

(21) Appl. No.: 18/562,733

(22) PCT Filed: Jun. 3, 2022
(86) PCT No.: PCT/IB2022/055214

§ 371 Date: Nov. 20, 2023

(30) Foreign Application Priority Data
Jun. 4, 2021 (IN) 202111024926

(51) Int. Cl. A61K 47/54; A61K 48/00; C07F 9/655; C12N 15/113

(52) U.S. Cl. CPC A61K 47/548; A61K 48/0033; C07F 9/65522; C12N 15/113

(57) ABSTRACT

There is provided a complex of a 6,7-dihydroxy coumarin phosphonium amphiphile together with a negatively charged agent. The complex self-assembles into a nanoparticle or non-viral vector for facilitating delivery of the negatively charged agent. The negatively charged agent may be a therapeutic agent or a diagnostic agent. Thus, the present invention provides a method of delivery of such negatively charged agents to a target cell, and in particular facilitates delivery of a therapeutic or diagnostic agent across the plasma membrane, as well as a method of treatment or diagnosis via delivery of the therapeutic or diagnostic agent. The 6,7-dihydroxy coumarin phosphonium amphiphile can be Mito-Esc and the negatively charged agent can be a nucleic acid such as siRNA. Additionally, the 6,7-dihydroxy coumarin phosphonium amphiphile is believed to be effective in the treatment of cancer.

### FIELD OF THE INVENTION

The present invention provides a novel delivery vector for nucleic acids such as plasmid DNAs, antisense oligonucleotides, small interfering RNAs (siRNAs), small hairpin RNAs (shRNAs), microRNA and messenger RNA to improve their use for the prevention or treatment of various diseases and/or disorders. Additionally, the present invention provides a novel treatment for cancer, such as breast cancer.

### BACKGROUND OF THE INVENTION

Nucleic acids have emerged as promising therapeutic candidates for cancer treatment, including immunotherapy. Nucleic acids are a diverse class of DNA or RNA such as plasmids, mRNA, ASO, siRNA, miRNA, small-activating RNA (saRNA), aptamers, gene-editing gRNA, as well as immunomodulatory DNA/RNA. Nucleic acid therapeutics have versatile functionalities ranging from altering (up- or down-regulating) gene expression, to modulating immune responses. The high specificity, versatile functionality, reproducible batch-to-batch manufacture, and tuneable immunogenicity of nucleic acids make them good candidates for cancer immunotherapy.

For example, small interfering RNAs (siRNAs) are emerging as novel and useful therapeutic agents to treat a wide range of medical conditions. The first such treatment approved by the US FDA is the drug patisiran (ONPATTRO™) which has been developed for the treatment of peripheral nerve disease (polyneuropathy) caused by hereditary transthyretin-mediated amyloidosis (hATTR) in adult patients, a rare, debilitating and often fatal genetic disease characterized by the build-up of abnormal amyloid protein in peripheral nerves, the heart and other organs. A further example of an FDA-approved siRNA treatment is GIVALAARI™ (givosiran) for the treatment of adult patients with acute hepatic porphyria, a genetic disorder resulting in the build-up of toxic porphyrin molecules which are formed during the production of heme (which helps bind oxygen in the blood). Other siRNA therapies are also in development and in clinical evaluation.

While siRNA has significant potential for treatment, challenges remain because introducing either naked or encapsulated nucleic acids into a carrier for combination with biological fluids encounters many physiological barriers that alter the cellular biodistribution and intracellular bioavailability of the siRNA. Following administration, unmodified DNA and RNA rapidly degrade in biological fluids by extra- and intracellular enzymes before they can reach the surface of the target cells. This influences their activity and interaction with the cells, compromising the therapeutic outcomes of nucleic acids. In addition, nucleic acids have very limited cellular uptake because of their hydrophilic nature and high molecular weight. A small fraction that can be taken up by the cells is usually internalized into vesicles (i.e., endosomes), which convert later into lysosomes. Accumulation and subsequent digestion of the nucleic acids inside the lysosomes precludes them from reaching their cytoplasmic or nuclear targets and is a significant barrier to their efficacy. For example, the limited stability of the siRNA, its immunogenicity and the challenge of delivering the siRNA therapeutic agent into the desired target site intracellularly, namely the difficulty of transporting the siRNAs across the plasma membrane into the cytoplasm of the cell are all barriers to siRNA efficacy. Many strategies have been investigated to increase the intracellular bioavailability of nucleic acids. These techniques mainly involve modifying the chemical structures of the nucleic acids or encapsulating the genetic materials into vectors (viral or non-viral). These vectors should be small enough to be taken up by cells and possess either targeting moieties or an excess positive charge to facilitate cell binding and subsequent uptake. In general, although a viral vector can achieve a higher degree of transport efficacy, concerns remain regarding the safety and limitations on scalability of such vectors. Accordingly, non-viral vectors are preferred despite the lower therapeutic effect. Incorporation of fusogenic lipids or peptides or membrane destabilizing polymers can be employed to facilitate escape of the genetic material from the endosomes/lysosomes into the cytoplasm. Tagging with a nuclear homing sequence to increase expression efficiency can also be utilized when using pDNA. Finally, these complexes should have intermediate stability; be robust enough to carry the nucleic acids to the target site, but dissociate from them at their targeted subcellular compartment/organelle. For example, due to the presence of the negatively charged phosphate groups within the RNA molecule, intracellular delivery to the target site requires the presence of a delivery vector. Inclusion of a delivery vector in the therapeutic composition, however, can limit the therapeutic potential of the active RNA agent.

Generally, non-viral vectors will be synthetic particles together with perpetually charged quaternary ammonium-based cationic lipids (PCCLs) or cationic polymers (PCCPs) and produce siRNA complexes followed by transfection, but generally require pH-responsive lipid comprising protonable amine groups to reduce toxicity. In this regard, tri-phenylphosphonium (TPP) tethered polymer-based delivery systems are gaining prominence as non-toxic alternatives to the ammonium-based systems because of their superior safety and transfection ability. Also, TPP-anchored molecules, due to the amphiphilic character and delocalized positive charge, exhibit enhanced biocompatibility, membrane fusion, cellular uptake and also mitochondrial targeting.

There remains a need for non-toxic nucleic acid delivery vectors able to provide a high transfection efficiency.

The present invention provides a novel nucleic acid delivery method based on a phosphonium amphiphile. Preferably, the phosphonium amphiphile described has the ability to form aggregates and subsequently deliver nucleic acids such as siRNA to the target cells by forming phosphonium-siRNA complexes. An exemplary phosphonium amphiphile according to the present invention is triphenylphosphoium cation (TPP+) coupled esculetin (herein referenced as “Mito-Esculetin” or “Mito-Esc”, which terms are used herein interchangeably).

The molecular structure of Mito-Esc consists of a lipophilic TPP cation linked to a hydrophilic 6,7-dihydroxy coumarin molecule through 8-carbon aliphatic chain. Thus, Mito-Esc is an amphiphilic molecule comprised of architecture possessing opposing faces, hydrophobic and hydrophilic groups, within the same molecule.

U.S. Pat. No. 9,580,452 describes the use of mito-esculetin for the treatment of atherosclerosis. PCT Application No. IB2020/061043 describes the use of mito-esculetin for the treatment of wounds, psoriasis and hair loss. Neither of these disclosures, however, suggests that mito-esculetin could be useful as a nucleic acid delivery vector, preferably as a siRNA delivery vector, nor that mito-esculetin could be useful for treatment of cancer, such as breast cancer.

In addition, recently it was demonstrated that mitochondria-targeted esculetin (Mito-Esc) greatly alleviates atherosclerotic disease progression by mitigating oxidant-induced endothelial dysfunction. Thereby showing that Mito-Esc, while improving the oxidant-mediated cellular abnormalities, causes preferential breast cancer cell death. With this background, the present invention exploited the hydrophobic property of the TPP cation and the hydrophilic property of 6,7-dihydroxy coumarin to form self-assembled nanoparticles and serve as an efficient nucleic acid delivery vector, such as a siRNA delivery vector.

### SUMMARY OF THE INVENTION

As a result of intensive studies, the present inventors have found that a 6,7-dihydroxy coumarin phosphonium amphiphile forms a complex together with a negatively charged agent. The negatively charged therapeutic agent may be a therapeutic agent or a diagnostic agent. The negatively charged agent can be a nucleic acid, for example plasmid DNAs, antisense oligonucleotides, small interfering RNAs (siRNAs), small hairpin RNAs (shRNAs), microRNA and messenger RNA (mRNA). More specifically a siRNA or mRNA, which is targeted at treating or diagnosing a specific disease or disorder.

Additionally, the present inventors have found that the complex of the 6,7-dihydroxy coumarin phosphonium amphiphile together with a negatively charged agent self-assembles into a nanoparticle or non-viral vector. Thus, the present invention provides a method of delivery of a negatively charged agent to a target cell, and in particular delivery of the agent across the plasma membrane.

Optionally, in the complex and nanoparticle of the present invention the 6,7-dihydroxy coumarin phosphonium amphiphile complex and nanoparticle is a compound of Formula I:

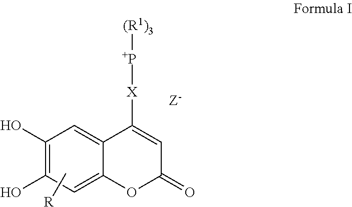

wherein,
Z is a negatively charged agent or a negatively charged counterion selected from a halide, mesylate, tosylate, citrate, tartrate, malate, acetate and trifluoroacetate;
R is hydrogen, a substituted alkyl, a substituted aryl or a substituted heteroatom;
R₁ is an aryl, a cycloalkyl or a heteroaryl;
X is a C₁ to C₃₀ carbon chain including one or more double or triple bonds, unsubstituted or substituted with alkyl, alkenyl or alkynyl side chains, or

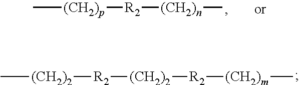

wherein,
p is 2 or 3, n is an integer from 3 to 6, and m is an integer from 2 to 4; and
R₂ is O, NH or S.

Preferably X is a C₆ to C₁₀ carbon chain, and R is hydrogen.

In one embodiment, in the complex and nanoparticle of the present invention the 6,7-dihydroxy coumarin phosphonium amphiphile is a triphenylphosphonium cation covalently coupled to a 6,7-dihydroxy coumarin moiety. More specifically, the complex and nanoparticle is a compound of Formula II:

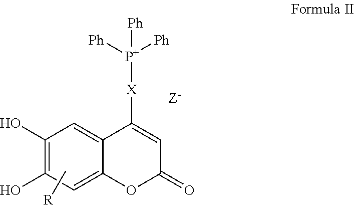

wherein,
X is a C₁ to C₃₀ carbon chain, preferably a C₆ to C₁₀ carbon chain;
Z is a negatively charged agent or a halide; and
R is hydrogen.

In some embodiments, the complex and nanoparticle of compound of formula I or II is a compound of Formula III:

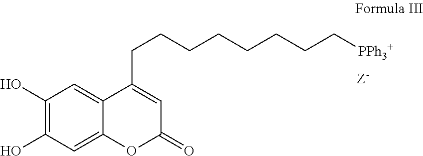

Z is a negatively charged agent or a halide.

Further studies have also shown that the 6,7-dihydroxy coumarin phosphonium amphiphile or pharmaceutically acceptable salt thereof is useful for the treatment or diagnosis of cancer. The present invention therefore also provides the 6,7-dihydroxy coumarin phosphonium amphiphile or pharmaceutically acceptable salt thereof for use in the treatment of cancer, in particular breast cancer and in a method of treatment of cancer, said method comprising administering the 6,7-dihydroxy coumarin phosphonium amphiphile or pharmaceutically acceptable salt thereof to a patient in need thereof.

The aforementioned aspects and embodiments, and other aspects, objects, features and advantages of the present invention will be apparent from the following detailed description.

### BRIEF DESCRIPTION OF THE DRAWINGS

FIG. 1: Mito-Esc dose- and time-dependently induces breast cancer cell death without affecting normal cell viability. (A) MDA-MB-231 breast cancer cells were treated with various concentrations of Mito-Esc (1.25-7.5 μM). Cell viability was measured by trypan blue dye exclusion assay at 24 h and 48 h. (B) Same as A, except that cells were treated with parent Esc (5-50 μM). (C) Same as A, except that MCF10A (normal mammary epithelial) cells were treated with Mito-Esc (5-50 μM). (*, Significantly different from control (P<0.05). ns; not significantly different from control).

FIG. 2: (A) DLS: Size distribution of Mito-Esc nanoparticles; (B) SEM image of Mito-Esc nanoparticles: Scanning electron micrographs of Mito-Esc nanoparticle (Scale bar 100 nm); (C) TEM image of Mito-ESC nanoparticle: Transmission electron micrographs (TEM) of Mito-Esc nanoparticles, samples were negatively stained with ammonium molyb-date (Scale bar 200 nm). (D) & (E) Binding ability of Mito-Esc: Agarose Gel electrophoresis assay of siRNA lipoplexes formed with Mito-Esc, and Mito-isoscopoletin and octyl TPP at different P+/P− ratio.

FIG. 3: MitoEsc administration regress breast cancer progression SCID mice model: (A) Representative tumors from each group are shown as indicated; (B) graph depicts mean tumor volume±SEM in mm³ on the day of termination; (C) graph shows tumor weights; (D) Mean Necrotic index was calculated in three individual tumor sections. Data represents Mean±SE of four animals.

FIG. 4: Mito-Esc/siMnSOD complex by depleting MnSOD levels exacerbates Mito-Esc-induced breast cancer cell death. (A) MDA-MB-231 cells were incubated either with Mito-Esc/siMnSOD (40 nM) complex or with lipofectamine-2000/siMnSOD complex for 48 h and cell viability was measured by trypan blue dye exclusion assay. (B) MDA-MB-231 cells were transfected with siMnSOD (40 nM) with indicated delivery systems for 48 h and MnSOD protein levels were measured by immunoblotting. Parenthesis indicates the relative expression of MnSOD normalized to GAPDH (loading control). (*, Significantly different from control (P<0.05). ns; not significantly different from control.).

FIG. 5: Detection of intracellular delivery of Cy-5 siRNA by Mito-Esc using confocal imaging. MDA-MB-231 cells were transfected with Cy-5 labelled siRNA (40 nM) with indicated delivery systems for 24 h. Transfection efficacy was evaluated using Confocal Microscopy. Cy-5 fluorescence in the cytosol is shown in blue color (Scale bar 10X).

FIG. 6: Comparison of intracellular delivery of Cy-5 siRNA using confocal imaging. MCF-10A cells were transfected with Cy-5 labelled siRNA using different delivery mechanisms. Transfection efficacy was evaluated using Confocal Microscopy. Cy-5 fluorescence in the cytosol is shown in blue.

### DETAILED DESCRIPTION OF THE INVENTION

As used herein the following definitions apply unless clearly indicated otherwise. It should be understood that unless expressly stated to the contrary, the singular forms “a” “an” and “the” include plural reference unless the context clearly dictates otherwise.

By “alkyl,” in the present invention is meant a straight or branched hydrocarbon radical and includes, for example, methyl, ethyl, n-propyl, isopropyl, n-butyl, Sec-butyl, isobutyl, tert-butyl, n-pentyl, iso-pentyl, n-hexyl, n-octyl and the like.

“Alkenyl’ means straight and branched hydrocarbon radicals and at least one double bond and includes, but is not limited to, ethenyl, 3-buten1-yl, 2-ethenylbutyl, 3-hexen-1-yl, and the like.

“Alkynyl’ means straight and branched hydrocarbon radicals and at least one triple bond and includes, but is not limited to, ethynyl, 3-butyn 1-yl, propynyl, 2-butyn-1-yl, 3-pentyn-1-yl, and the like.

By “aryl’ is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which can be mono-, di-, or trisubstituted with, e.g., halogen, lower alkyl, lower alkoxy, lower alkylthio, trifluoromethyl, lower acyloxy, aryl, heteroaryl, and hydroxy. A preferred aryl is phenyl.

“Heteroatom”, means an atom of any element other than carbon or hydrogen. Preferably, heteroatoms are nitrogen, oxygen, and sulfur.

“Cycloalkyl” means a monocyclic or polycyclic hydrocarbyl group having from 3 to 8 carbon atoms, for instance, cyclopropyl, cycloheptyl, cyclooctyl, cyclodecyl, cyclobutyl, adamantyl, norpinanyl, decalinyl, norbornyl, cyclohexyl, and cyclopentyl.

By the term “halide’ in the present invention is meant fluoride, bromide, chloride, and iodide.

By “heteroaryl’ is meant one or more aromatic ring systems of 5-, 6-, or 7-membered rings containing at least one and up to four heteroatoms selected from nitrogen, oxygen, or sulfur. Such heteroaryl groups include, for example, thienyl, furanyl, thiazolyl, triazolyl, imidazolyl, (is)oxazolyl, oxadiazolyl, tetrazolyl, pyridyl, thiadiazolyl, oxadiazolyl, oxathiadiazolyl, thiatriazolyl, pyrimidinyl, (iso) quinolinyl, napthyridinyl, phthalimidyl, benzimidazolyl, and benzoxazolyl.

“Therapeutically effective amount” as used herein refers to the amount of a therapeutic agent that is effective to alleviate the target disease or disorder.

“Patient” as used herein refers to any human or nonhuman animal (e.g., primates, sheep, dogs, cats, horses, cows, chickens, amphibians, reptiles and the like). As used herein the term “Treatment” refers to cure the disease and/or disorder as rapidly as possible and to prevent the progression to severe disease.

The present invention is based on the surprising finding that a 6,7-dihydroxy coumarin phosphonium amphiphile can self-aggregate with a negatively charged agent, to form a vector or nanoparticle which is particularly suitable to facilitate the delivery of the agent into a target cell (for example a cancer cell). The negatively charged agent may be a therapeutic agent or a diagnostic agent. Preferably, the 6,7-dihydroxy coumarin phosphonium amphiphile is a triphenylphosphonium cation covalently coupled to a 6,7-dihydroxy coumarin moiety. A preferred 6,7-dihydroxy coumarin phosphonium amphiphile is octyl tagged esculetin (Mito-Esculetin). Additionally, studies have further demonstrated that the 6,7-dihydroxy coumarin phosphonium amphiphile is useful for the treatment and diagnosis of cancer, in particular breast cancer, cervical cancer, lung cancer and liver cancer. More particularly, in breast cancer such as triple-negative breast cancer and ER+ve breast cancer.

In one aspect, the present invention provides complex of a 6,7-dihydroxy coumarin phosphonium amphiphile together with a negatively charged agent.

The negatively charged agent can be a nucleic acid, for example, a plasmid DNAs, antisense oligonucleotides, small interfering RNAs (siRNAs), small hairpin RNAs (shRNAs), microRNA and messenger RNA (mRNA). However, all that is required is that the agent possesses a negative charge, so that it will associate with the 6,7-dihydroxy coumarin phosphonium amphiphile. The negatively charged agent may be a therapeutic agent or a diagnostic agent. In some embodiments, the therapeutic agent is intended for delivery to the cytoplasm of a target cell, thereby exerting its intended therapeutic effect within the cytoplasm of the target cell. In some embodiments, the therapeutic agent will be negatively charged anti-cancer agents, a siRNA or mRNA. The siRNA may be effective for the treatment or diagnosis of any disease or disorder, for example the treatment or diagnosis of cancer, peripheral nerve disease, acute hepatic porphyria and others. In some embodiments, the siRNA can be used to treat breast cancer, cervical cancer, lung cancer and liver cancer. More particularly, breast cancers such as triple-negative breast cancer and ER+ve breast cancer.

Alternatively, the therapeutic agent can be an RNA vaccine, for example an RNA vaccine against a virus, for example coronavirus.

In one embodiment, the 6,7-dihydroxy coumarin phosphonium amphiphile complex is a compound of Formula I

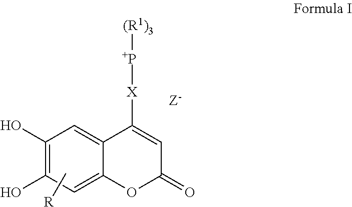

wherein,
Z is a negatively charged agent or a negatively charged counterion selected from a halide, mesylate, tosylate, citrate, tartrate, malate, acetate and trifluoroacetate;
R is hydrogen, a substituted alkyl, a substituted aryl or a substituted heteroatom;
R₁ is an aryl, a cycloalkyl or a heteroaryl;
X is a C₁ to C₃₀ carbon chain including one or more double or triple bonds, unsubstituted or substituted with alkyl, alkenyl or alkynyl side chains, or

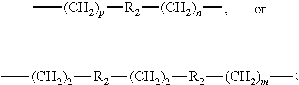

wherein,
p is 2 or 3, n is an integer from 3 to 6, and m is an integer from 2 to 4; and
R₂ is O, NH or S.

In another embodiment, the 6,7-dihydroxy coumarin phosphonium amphiphile can be a triphenylphosphonium cation covalently coupled to a 6,7-dihydroxy coumarin moiety. More specifically, the 6,7-dihydroxy coumarin phosphonium amphiphile complex is a compound of Formula II:

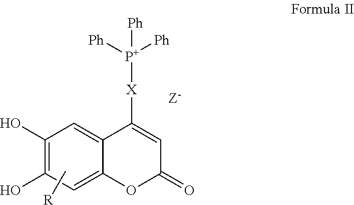

wherein,
X is a C₁ to C₃₀ carbon chain, preferably a C₆ to C₁₀ carbon chain;
Z is a negatively charged agent or a halide; and
R is hydrogen, one or more substituted alkyl, one or more substituted aryl or one or more substituted heteroatom. Preferably, R is hydrogen.

In one embodiment, the negatively charged agent may be a therapeutic agent or a diagnostic agent. Preferably, Z is a negatively charged therapeutic agent.

In one embodiment, X is a C1 to C30 carbon chain including one or more double or triple bonds, unsubstituted or substituted with alkyl, alkenyl or alkynyl side chains. Preferably, X is an octylene group.

In some embodiments, the 6,7-dihydroxy coumarin phosphonium amphiphile is Mito-Esculetin. More specifically, the 6,7-dihydroxy coumarin phosphonium amphiphile complex is a compound of formula III:

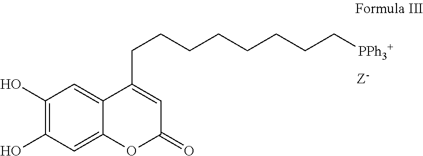

Z is a negatively charged agent or a halide.

U.S. Pat. No. 9,580,452 describes a method of synthesis of compounds according to Formula I, II and III, particular the synthesis of Mito-Esculetin.

Optionally, the complex comprises Mito-Esculetin in combination with an RNA therapeutic agent for example a siRNA.

It was surprisingly found that the complexes of the invention can self-assemble into nanoparticles or non-viral vectors. These nanoparticles are particularly suitable for delivery of a therapeutic agent or diagnostic agent to a patient, and in particular are suitable for delivery of a negatively charged therapeutic agent into the cytoplasm of a target cell.

Accordingly, the present invention provides, in addition to the complex described above, a nanoparticle comprising a 6,7-dihydroxy coumarin phosphonium amphiphile.

The nanoparticle preferably comprises a negatively charged agent. The negatively charged agent can be a nucleic acid, for example, plasmid DNAs, antisense oligonucleotides, small interfering RNAs (siRNAs), small hairpin RNAs (shRNAs), microRNA and messenger RNA (mRNA). However, all that is required is that the agent possesses a negative charge, so that it will associate with the 6,7-dihydroxy coumarin phosphonium amphiphile. The negatively charged agent may be a therapeutic agent or a diagnostic agent. In some embodiments, the therapeutic agent is intended for delivery to the cytoplasm of a target cell, thereby exerting its intended therapeutic effect within the cytoplasm of the target cell. In some embodiments, the therapeutic agent will be negatively charged anti-cancer agents, a siRNA or mRNA. The siRNA may be effective for the treatment or diagnosis of any disease or disorder, for example the treatment or diagnosis of cancer, peripheral nerve disease, acute hepatic porphyria and others. In some embodiments, the siRNA can be used to treat breast cancer, cervical cancer, lung cancer and liver cancer. More particularly, breast cancers such as triple-negative breast cancer and ER+ve breast cancer. Alternatively, the therapeutic agent can be an RNA vaccine, for example an RNA vaccine against a virus, for example coronavirus. In some embodiments, the agent is a siRNA. The siRNA can be targeted to treating or diagnosing any specific disease or disorder.

In some embodiments, the nanoparticle can have a size of 100 to 200 nm, for example from 150 to 180 nm, preferably around 160-170 nm.

In some embodiments, the nanoparticle can have a surface charge of 30 to 40 mV.

In some embodiments, the nanoparticle can have a surface charge of 30 mV or greater.

The present invention further provides a composition comprising a nanoparticle or complex according to the present invention. Optionally, the composition is an aqueous solution or suspension.

In some embodiments, the composition according to the invention is in a pharmaceutically acceptable form.

In certain embodiments, the pharmaceutical composition is formulated for oral or parenteral administration. In some embodiments, the pharmaceutical composition is administered as an oral dosage form. Preferably, the oral dosage form is in the form of tablet, capsule, dispersible tablets, sachets, sprinkles, liquids, solution, suspension, emulsion and the like. If the oral dosage form is a tablet, the tablet can be of any suitable shape such as round, spherical, or oval. The tablet may have a monolithic or a multi-layered structure. In some embodiments, the pharmaceutical composition of the present invention can be obtained by conventional approaches using conventional pharmaceutically acceptable excipients well known in the art. Examples of pharmaceutically acceptable excipients suitable for tablet preparation include, but are not limited to, diluents (e.g., calcium phosphate-dibasic, calcium carbonate, lactose, glucose, microcrystalline cellulose, cellulose powdered, silicified microcrystalline cellulose, calcium silicate, starch, starch pregelatinized, or polyols such as mannitol, sorbitol, xylitol, maltitol, and sucrose), binders (e.g., starch, pregelatinized starch, carboxymethyl cellulose, sodium cellulose, microcrystalline cellulose, hydroxypropyl cellulose, hydroxypropyl methylcellulose, polyvinylpyrrolidone, crospovidone, or combinations thereof), disintegrants (e.g., cross-linked cellulose, cross-linked-polyvinylpyrrolidone (crosspovidone), sodium starch glycolate, polyvinylpyrrolidone (polyvidone, povidone), sodium carboxymethylcellulose, cross-linked sodium carboxymethylcellulose (croscarmellose sodium), hydroxypropyl cellulose, hydroxypropyl methylcellulose, xanthan gum, alginic acid, or soy polysaccharides), wetting agents (e.g., polysorbate, sodium lauryl sulphate, or glyceryl stearate) or lubricants (e.g., sodium lauryl sulfate, talc, magnesium stearate, sodium stearyl fumarate, stearic acid, glyceryl behenate, hydrogenated vegetable oil, or zinc stearate). The tablets so prepared may be uncoated or coated for altering their disintegration, and subsequent enteral absorption of the active ingredient, or for improving their stability and/or appearance. In both cases, conventional coating agents and approaches well known in the art can be employed.

In certain embodiments, the parenteral administration can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and about 1-10% human serum albumin. Liposomes and non-aqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques. In some embodiments, parenteral formulation may comprise a common excipient that includes, but not limited to, sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Parenteral route of administration includes, but is not limited to, subcutaneous route, intramuscular route, intravenous route, intrathecal route or intraperitoneal.

The formulations of the present invention can be prepared by a process known or otherwise described in the prior art, for example the process disclosed in Remington's Pharmaceutical Sciences.

Optionally, the complex or nanoparticle of the present invention can be useful in the treatment or diagnosis of cancer. Preferably, for example, for the treatment of breast cancer, cervical cancer, lung cancer and liver cancer. More particularly, in breast cancers such as triple-negative breast cancer and ER+ve breast cancer.

The present invention further provides a method of delivering a negatively charged agent, wherein said agent is complexed to a 6,7-dihydroxy coumarin phosphonium amphiphile. Optionally the complex of the agent and 6,7-dihydroxy coumarin phosphonium amphiphile is in the form of a nanoparticle, as described above. The negatively charged agent may be a therapeutic agent or a diagnostic agent. Preferably, the present invention provides a method of delivering a negatively charged therapeutic agent, wherein said agent is complexed to a 6,7-dihydroxy coumarin phosphonium amphiphile. More preferably, the complex of the therapeutic agent and 6,7-dihydroxy coumarin phosphonium amphiphile is in the form of a nanoparticle.

The present invention further provides a method of intracellular delivery of a negatively charged agent, said method comprising administering an effective amount of said agent complexed to a 6,7-dihydroxy coumarin phosphonium amphiphile. Optionally, the complex of the agent and 6,7-dihydroxy coumarin phosphonium amphiphile is in the form of a nanoparticle, as described above. The negatively charged agent may be a therapeutic agent or a diagnostic agent. Preferably, the invention provides a method of intracellular delivery of a negatively charged therapeutic agent, said method comprising administering an effective amount of said therapeutic agent complexed to a 6,7-dihydroxy coumarin phosphonium amphiphile. More preferably, the complex is in the form of a nanoparticle.

In the methods described above, the negatively charged agent is complexed to a 6,7-dihydroxy coumarin phosphonium amphiphile, and said complex can be further assembled into the form of a nanoparticle or non-viral vector. The negatively charged agent can be a nucleic acid, for example, plasmid DNAs, antisense oligonucleotides, small interfering RNAs (siRNAs), small hairpin RNAs (shRNAs), microRNA and messenger RNA (mRNA). However, all that is required is that the agent possesses a negative charge, so that it will associate with the 6,7-dihydroxy coumarin phosphonium amphiphile. The negatively charged agent may be a therapeutic agent or a diagnostic agent. In some embodiments, the therapeutic agent is intended for delivery to the cytoplasm of a target cell, thereby exerting its intended therapeutic effect within the cytoplasm of the target cell. In some embodiments, the therapeutic agent will be negatively charged anti-cancer agent, a siRNA or mRNA. The siRNA may be effective for the treatment or diagnosis of any disease or disorder, for example, the treatment or diagnosis of cancer, peripheral nerve disease, acute hepatic porphyria and others. In some embodiments, the siRNA can be used to treat breast cancer, cervical cancer, lung cancer and liver cancer. More particularly, in breast cancers such as triple-negative breast cancer and ER+ve breast cancer. Alternatively, the therapeutic agent can be an RNA vaccine, for example an RNA vaccine against a virus, for example coronavirus. In some embodiments, the agent is a siRNA. The siRNA can be targeted at treating or diagnosing any specific disease or disorder. For example, the siRNA can be effective at targeting cancer to trigger their specific cell death and/or to prevent cell growth and division of such cells. For example, the siRNA can be specifically targeted to breast cancer cells.

Thus, the present invention also provides a method of treating or ameliorating the progression of cancer, wherein said method comprises administration of a pharmaceutically acceptable composition comprising the nanoparticle of 6,7-dihydroxy coumarin phosphonium amphiphile and negatively charged therapeutic agent to the patient.

In one embodiment, said 6,7-dihydroxy coumarin phosphonium amphiphile is Mito-Esc and said therapeutic agent is a siRNA which is therapeutically effective against the cancer.

In a further aspect, the present invention provides a 6,7-dihydroxy coumarin phosphonium amphiphile or pharmaceutically acceptable salt thereof for use in the treatment or diagnosis of cancer. Optionally, the cancer is breast cancer.

The 6,7-dihydroxy coumarin phosphonium amphiphile for use in the treatment or diagnosis of cancer can be a compound of Formula I:

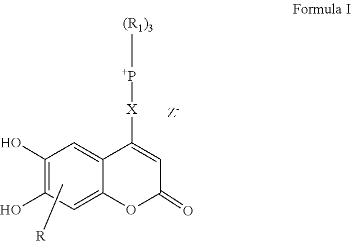

wherein,
Z is a negatively charged agent or a negatively charged counterion selected from a halide, mesylate, tosylate, citrate, tartrate, malate, acetate and trifluoroacetate;
R is hydrogen, a substituted alkyl, a substituted aryl or a substituted heteroatom;
R₁ is an aryl, a cycloalkyl or a heteroaryl;
X is a C₁ to C₃₀ carbon chain including one or more double or triple bonds, unsubstituted or substituted with alkyl, alkenyl or alkynyl side chains, or

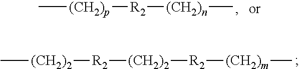

wherein,
p is 2 or 3, n is an integer from 3 to 6, and m is an integer from 2 to 4; and
R₂ is O, NH or S.

In another embodiment, the 6,7-dihydroxy coumarin phosphonium amphiphile for use in the treatment or diagnosis of cancer can be a triphenylphosphonium cation covalently coupled to a 6,7-dihydroxy coumarin moiety. More specifically, the 6,7-dihydroxy coumarin phosphonium amphiphile is a compound of formula II:

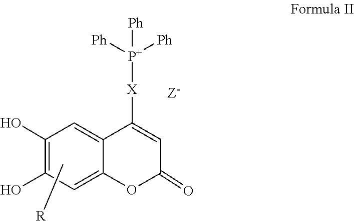

wherein,
X is a C₁ to C₃₀ carbon chain, preferably a C₆ to C₁₀ carbon chain;
Z is a negatively charged agent or a negatively charged counterion selected from a halide, mesylate, tosylate, citrate, tartrate, malate, acetate and trifluoroacetate;
R is hydrogen, a substituted alkyl, a substituted aryl, or a substituted heteroatom. Preferably, R is hydrogen.

In one embodiment, X is a C1 to C30 carbon chain including one or more double or triple bonds, unsubstituted or substituted with alkyl, alkenyl or alkynyl side chains. Preferably, X is an octylene group.

In one embodiment, the negatively charged agent may be a therapeutic agent or a diagnostic agent. Optionally, Z is a bromide anion. Optionally, Z is a negatively charged therapeutic agent, for example, a siRNA, suitable to target the cancer being treated. In some embodiments, the cancer is breast cancer.

In some embodiments, the 6,7-dihydroxy coumarin phosphonium amphiphile for use in the treatment of cancer is Mito-Esc of the formula III:

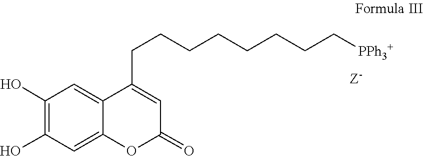

Z is a negatively charged agent or a negatively charged counterion selected from a halide, mesylate, tosylate, citrate, tartrate, malate, acetate and trifluoroacetate.

In a further aspect, the present invention provides a method of treating or diagnosing cancer, for example, breast cancer, cervical cancer, lung cancer and liver cancer. More particularly, a method of treating breast cancer such as triple-negative breast cancer and ER+ve breast cancer, said method comprising administering an effective amount of a compound of formula I to a patient in need thereof, wherein said compound is a compound of formula II:

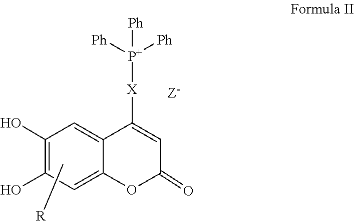

wherein,
X is a C₁ to C₃₀ carbon chain, preferably a C₆ to C₁₀ carbon chain;
Z is a negatively charged therapeutic agent or a negatively charged counterion selected from a halide, mesylate, tosylate, citrate, tartrate, malate, acetate and trifluoroacetate;
R is hydrogen, a substituted alkyl, a substituted aryl or a substituted heteroatom. Preferably, R is hydrogen.

In one embodiment, X is a C1 to C30 carbon chain including one or more double or triple bonds, unsubstituted or substituted with alkyl, alkenyl or alkynyl side chains. Preferably, X is an octylene group.

Optionally, Z is a bromide anion. Optionally, Z is a negatively charged therapeutic agent, for example, siRNA suitable to target the cancer being treated. In some embodiments, the cancer is breast cancer.

Preferably, the compound of Formula I or Formula II is Mito-Esc of the formula III:

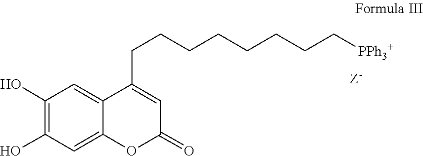

wherein Z is a negatively charged agent or a halide.

In one aspect, the present disclosure relates to a complex of 6,7-dihydroxy coumarin phosphonium amphiphile and a negatively charged agent.

In another aspect, the present disclosure relates to a nanoparticle comprising a complex of 6,7-dihydroxy coumarin phosphonium amphiphile and a negatively charged agent.

In one embodiment of the present disclosure, the 6,7-dihydroxy coumarin phosphonium amphiphile is a compound of Formula IV:

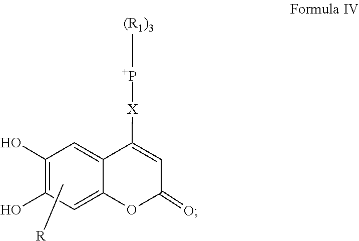

wherein,
R is hydrogen, a substituted alkyl, a substituted aryl or a substituted heteroatom;
R₁ is an aryl, a cycloalkyl or a heteroaryl;
X is a C₁ to C₃₀ carbon chain including one or more double or triple bonds, unsubstituted or substituted with alkyl, alkenyl or alkynyl side chains, or

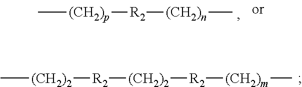

wherein,
p is 2 or 3, n is an integer from 3 to 6, and m is an integer from 2 to 4; and
R₂ is O, NH or S.

In an alternate embodiment, the 6,7-dihydroxy coumarin phosphonium amphiphile is a triphenylphosphonium cation covalently coupled 6,7-dihydroxy coumarin moiety of Formula V:

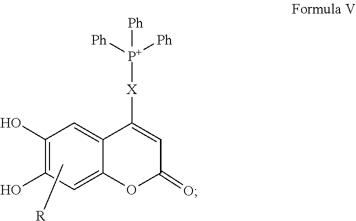

wherein,
R is hydrogen, a substituted alkyl, a substituted aryl or a substituted heteroatom;
R₁ is an aryl, a cycloalkyl or a heteroaryl;
X is a C₁ to C₃₀ carbon chain including one or more double or triple bonds, unsubstituted or substituted with alkyl, alkenyl or alkynyl side chains, or

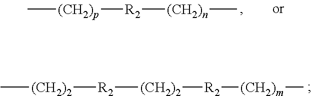

wherein,
p is 2 or 3, n is an integer from 3 to 6, and m is an integer from 2 to 4; and
R₂ is O, NH or S.

In yet alternate embodiment, the 6,7-dihydroxy coumarin phosphonium amphiphile is octyl tagged esculetin (Mito-Esculetin/Mito-Esc) of Formula VI:

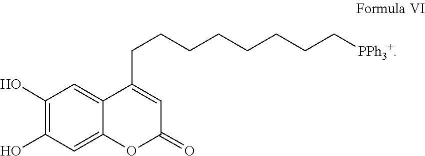

In another embodiment, the complex of 6,7-dihydroxy coumarin phosphonium amphiphile and a negatively charged agent is represented by a compound of Formula I:

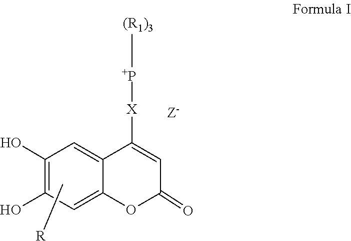

wherein,
Z is a negatively charged agent or a negatively charged counterion selected from mesylate, tosylate, citrate, tartrate, malate, acetate and trifluoroacetate;
R is hydrogen, a substituted alkyl, a substituted aryl or a substituted heteroatom;
R₁ is an aryl, a cycloalkyl or a heteroaryl;
X is a C₁ to C₃₀ carbon chain including one or more double or triple bonds, unsubstituted or substituted with alkyl, alkenyl or alkynyl side chains, or

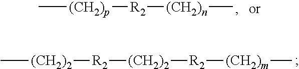

wherein,
p is 2 or 3, n is an integer from 3 to 6, and m is an integer from 2 to 4; and
R₂ is O, NH or S.

In an alternate embodiment, the complex of 6,7-dihydroxy coumarin phosphonium amphiphile and a negatively charged agent is represented by a compound of Formula II:

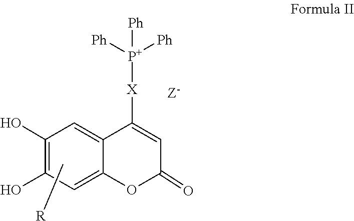

wherein,
X is a C₁ to C₃₀ carbon chain, preferably a C₆ to C₁₀ carbon chain;
Z is a negatively charged agent or a negatively charged counterion selected from mesylate, tosylate, citrate, tartrate, malate, acetate and trifluoroacetate; and
R is hydrogen, one or more substituted alkyl, one or more substituted aryl or one or more substituted heteroatom. Preferably, R is hydrogen.

In yet alternate embodiment, the complex of 6,7-dihydroxy coumarin phosphonium amphiphile and a negatively charged agent is represented by a compound of Formula III:

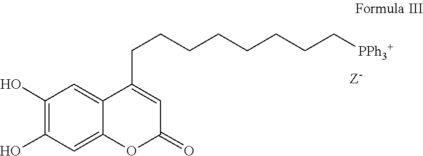

wherein,
Z is a negatively charged agent or a negatively charged counterion selected from mesylate, tosylate, citrate, tartrate, malate, acetate and trifluoroacetate.

In a preferred embodiment, Z is a negatively charged agent selected from a therapeutic agent, a diagnostic agent and a nucleic acid.

In yet another embodiment, the negatively charged agent can be a nucleic acid, for example, a plasmid DNAs, antisense oligonucleotides, small interfering RNAs (siRNAs), small hairpin RNAs (shRNAs), microRNA and messenger RNA (mRNA). However, all that is required is that the agent possesses a negative charge, so that it will associate with the 6,7-dihydroxy coumarin phosphonium amphiphile. The negatively charged agent may be a therapeutic agent or a diagnostic agent. In some embodiments, the therapeutic agent is intended for delivery to the cytoplasm of a target cell, thereby exerting its intended therapeutic effect within the cytoplasm of the target cell. In some embodiments, the therapeutic agent will be negatively charged anti-cancer agents, a siRNA or mRNA. The siRNA may be effective for the treatment or diagnosis of any disease or disorder, for example, the treatment or diagnosis of cancer, peripheral nerve disease, acute hepatic porphyria and others. In some embodiments, the siRNA can be used to treat breast cancer, cervical cancer, lung cancer and liver cancer. More particularly, breast cancers such as triple-negative breast cancer and ER+ve breast cancer.

Alternatively, the therapeutic agent can be an RNA vaccine, for example, an RNA vaccine against a virus, for example, coronavirus. In some embodiments, the agent is a siRNA. The siRNA can be targeted at treating or diagnosing any specific disease or disorder. For example, the siRNA can be effective at targeting cancer to trigger their specific cell death and/or to prevent cell growth and division of such cells. For example, the siRNA can be specifically targeted to breast cancer cells.

In a preferred embodiment, the complex of 6,7-dihydroxy coumarin phosphonium amphiphile and a negatively charged agent is a complex of Mito-Esc and siRNA, wherein Mito-Esc is represented by a compound of Formula VI:

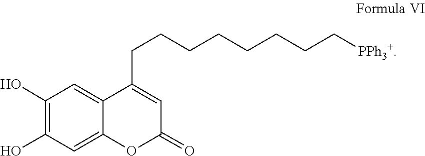

In another preferred embodiment, the nanoparticle comprising complex of 6,7-dihydroxy coumarin phosphonium amphiphile and a negatively charged agent is a nanoparticle comprising a complex of Mito-Esc and siRNA, wherein Mito-Esc is represented by a compound of Formula VI:

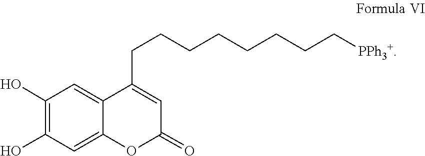

Optionally, the complex or nanoparticle of the present disclosure is useful in the treatment or diagnosis of cancer. Preferably, for example, for the treatment of breast cancer, cervical cancer, lung cancer and liver cancer. More particularly, in breast cancers such as triple-negative breast cancer and ER+ve breast cancer.

The present disclosure further provides a pharmaceutical composition comprising a nanoparticle or a complex according to the aspects of present disclosure.

The present disclosure also provides a method of treating or ameliorating the progression of cancer, wherein said method comprises administration of a pharmaceutical composition comprising the nanoparticle or the complex of 6,7-dihydroxy coumarin phosphonium amphiphile and negatively charged therapeutic agent to the patient.

In certain embodiments, the pharmaceutical composition is formulated for oral or parenteral administration. In some embodiments, the pharmaceutical composition is administered as an oral dosage form. Preferably, the oral dosage form is in the form of tablet, capsule, dispersible tablets, sachets, sprinkles, liquids, solution, suspension, emulsion and the like. If the oral dosage form is a tablet, the tablet can be of any suitable shape such as round, spherical, or oval. The tablet may have a monolithic or a multi-layered structure. In some embodiments, the pharmaceutical composition of the present invention can be obtained by conventional approaches using conventional pharmaceutically acceptable excipients well known in the art. Examples of pharmaceutically acceptable excipients suitable for tablet preparation include, but not limited to, diluents (e.g., calcium phosphate-dibasic, calcium carbonate, lactose, glucose, microcrystalline cellulose, cellulose powdered, silicified microcrystalline cellulose, calcium silicate, starch, starch pregelatinized, or polyols such as mannitol, sorbitol, xylitol, maltitol, and sucrose), binders (e.g., starch, pregelatinized starch, carboxymethyl cellulose, sodium cellulose, microcrystalline cellulose, hydroxypropyl cellulose, hydroxypropyl methylcellulose, polyvinylpyrrolidone, crospovidone, or combinations thereof), disintegrants (e.g., cross-linked cellulose, cross-linked-polyvinylpyrrolidone (crosspovidone), sodium starch glycolate, polyvinylpyrrolidone (polyvidone, povidone), sodium carboxymethylcellulose, cross-linked sodium carboxymethylcellulose (croscarmellose sodium), hydroxypropyl cellulose, hydroxypropyl methylcellulose, xanthan gum, alginic acid, or soy polysaccharides), wetting agents (e.g., polysorbate, sodium lauryl sulphate, or glyceryl stearate) or lubricants (e.g., sodium lauryl sulfate, talc, magnesium stearate, sodium stearyl fumarate, stearic acid, glyceryl behenate, hydrogenated vegetable oil, or zinc stearate). The tablets so prepared may be uncoated or coated for altering their disintegration, and subsequent enteral absorption of the active ingredient, or for improving their stability and/or appearance. In both cases, conventional coating agents and approaches well known in the art can be employed.

In certain embodiments, the parenteral administration can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and about 1-10% human serum albumin. Liposomes and non-aqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques. In some embodiments, parenteral formulation may comprise a common excipient that includes, but not limited to, sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Parenteral route of administration includes, but not limited to subcutaneous route, intramuscular route, intravenous route, intrathecal route or intraperitoneal.

The formulations of the present invention can be prepared by a process known or otherwise described in the prior art, for example the process disclosed in Remington's Pharmaceutical Sciences.

The present invention further provides a method of delivering a negatively charged agent, wherein said agent is complexed to a 6,7-dihydroxy coumarin phosphonium amphiphile. Optionally, the complex of the agent and 6,7-dihydroxy coumarin phosphonium amphiphile is in the form of a nanoparticle, as described above. The negatively charged agent may be a therapeutic agent or a diagnostic agent. Preferably, the present invention provides a method of delivering a negatively charged therapeutic agent, wherein said agent is complexed to a 6,7-dihydroxy coumarin phosphonium amphiphile. More preferably, the complex of the therapeutic agent and 6,7-dihydroxy coumarin phosphonium amphiphile is in the form of a nanoparticle.

The present invention further provides a method of intracellular delivery of a negatively charged agent, said method comprising administering an effective amount of said agent complexed to a 6,7-dihydroxy coumarin phosphonium amphiphile. Optionally, the complex of the agent and 6,7-dihydroxy coumarin phosphonium amphiphile is in the form of a nanoparticle, as described above. The negatively charged agent may be a therapeutic agent or a diagnostic agent. Preferably, the invention provides a method of intracellular delivery of a negatively charged therapeutic agent, said method comprising administering an effective amount of said therapeutic agent complexed to a 6,7-dihydroxy coumarin phosphonium amphiphile. More preferably, the complex is in the form of a nanoparticle.

The present invention is illustrated below by reference to the following examples. However, one skilled in the art will appreciate that specific methods and results discussed are merely illustrative of the invention, as innumerable variations, modifications, applications, and extensions of these embodiments and principles can be made without departing from the spirit and scope of the invention.

### EXAMPLES

### Example 1: Synthesis of Compounds

Synthesis of Mito-Esculetin and control TPP molecules was carried according to the following synthetic protocols.

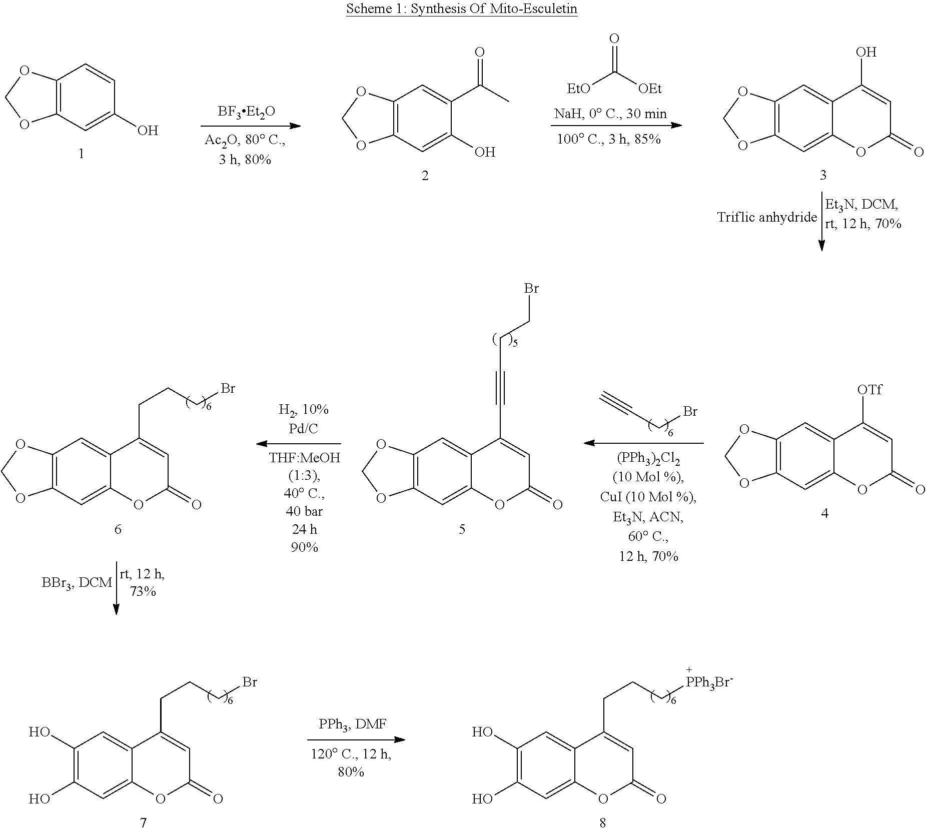

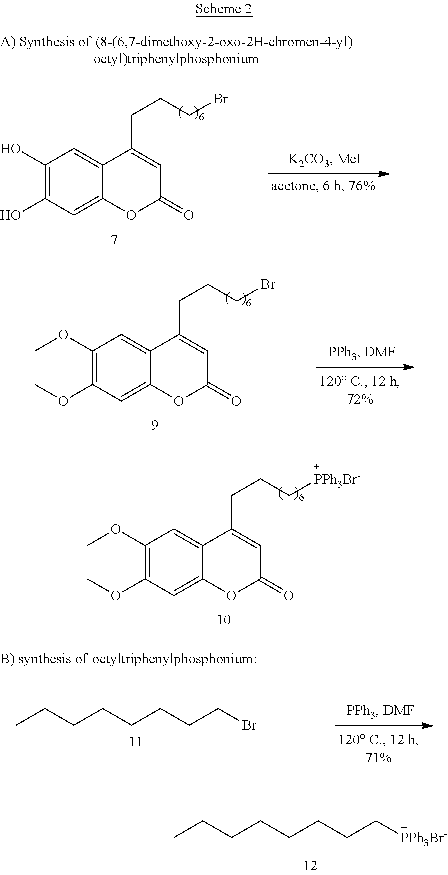

### Procedure for the Synthesis of 1-(6-hydroxybenzo[d][1,3]dioxol-5-yl)ethan-1-one (2)

A solution of sesamol (5.6 g, 40 mmol) in acetic anhydride (20 mL) was cooled to 0° C. under nitrogen atmosphere. The solution was slowly added with boron trifluoride/diethyl ether complex (10 mL), and then the mixture was stirred at 90° C. for 3 hours. The resulting mixture was added to saturated aqueous sodium acetate (50 mL), and stirred at room temperature. The solid formed was removed by filtration, the solvent was evaporated under reduced pressure, and the residual solid was suspended in methanol, thereby washed, then collected by filtration and dried to obtain 2 (5.850 g, 80%).

### Procedure for the Synthesis of 8-hydroxy-6H-[1,3]dioxolo[4,5-g]chromen-6-one (3)

To a solution of 2 (5 g, 1 eq.) in diethyl carbonate (80 mL) under nitrogen atmosphere was added sodium hydride (2.66 g, 4 eq.), and the mixture was stirred for 30 min at 0° C. The resulting solution was heated at 100° C. for 3 h, and then cooled to 0° C. and 50% aqueous MeOH (10 mL) was cautiously added. After extraction with ether (3×100 mL), the reaction mixture was acidified to pH 2 with 2N hydrochloric acid, and the precipitated solid was filtered and dried under vacuum to obtain 3 (4.9 g, 85%).

### Procedure for the Synthesis of 6-oxo-6H-[1,3]dioxolo[4,5-g]chromen-8-yl trifluoromethanesulfonate (4)

Trifluoromethanesulfonic anhydride (4.3 mL, 1.3 equiv) was added dropwise over 10 min. to a mixture of 3 (4 g, 1 eq.) and triethylamine (3.5 mL, 1.3 equiv) in dry dichloromethane (30 mL) at 0° C. Then the mixture was stirred for 12 h at room temperature. After that, the mixture was diluted with 50% ether:hexane and filtered through a short pad of silica, the filtrate was concentrated to a residue, which was purified by flash chromatography to give the corresponding product 4 (4.6 g, 70%).

### Procedure for the Synthesis of 8-(8-bromooct-1-yn-1-yl)-6H-[1,3]dioxolo[4,5-g]chromen-6-one (5)

A round-bottom flask was flame-dried under high vacuum. Upon cooling, coumarin 4 (1.0 g, 1 eq.), PdCl2(PPh3)2 (207 mg, 0.1 eq.), CuI (56 mg, 0.1 eq.), acetonitrile (10 mL), triethylamine (0.61 mL, 1.5 equiv) and 8-bromooctyne (0.838 g, 1.5 eq.) were added. The reaction mixture was stirred overnight at 60° C. Following completion of the reaction (monitored by TLC), the reaction mixture was cooled, diluted with ethyl acetate (20 mL), and filtered through a short silica gel bed. The filtrate was concentrated to a residue which was purified by flash chromatography to give the corresponding product 5 (0.790 g, 70%).

### Procedure for the Synthesis of 8-(8-bromooctyl)-6H-[1,3]dioxolo[4,5-g]chromen-6-one (6)

A well stirred mixture of coumarin 5 (0.7 g) in methanol was allowed to pass through a H-Cube reactor packed with 10% Pd/C at 1 mL/min, 40° C., and pressure of 40 bar. After completion of the reaction, the solvent was evaporated under reduced pressure to give corresponding product 6 (0.641 g, 90%).

### Procedure for the Synthesis of 4-(8-bromooctyl)-6,7-dihydroxy-2H-chromen-2-one (7)

8-(8-Bromooctyl)-6H-[1,3]dioxolo[4,5-g]chromen-6-one 6 (0.6 g, 1.0 equiv) was dissolved in dry DCM (15 mL) in a 50 mL round-bottom flask and the mixture was cooled to −78° C. BBr3 (1.0 M in DCM, 4 eq.) was added slowly dropwise. The reaction was allowed to warm to room temperature and stirred for 12 h. MeOH (2 mL) was added, with subsequent stirring for another 15 minute and the solvent was removed under vacuum. The crude product was purified by column chromatography on silica gel to afford 7 (0.425 g, 73%) as a yellow solid.

### Procedure for the Synthesis of (8-(6,7-dihydroxy-2-oxo-2H-chromen-4-yl)octyl) triphenylphosphonium (8)

To a stirred solution of compound 7 (0.2 g, 1 eq.) in dry DMF (6 ml) was added triphenylphosphine (0.156 g, 1.1 eq.) and the resulting mixture was heated to 120° C. for 12 h under nitrogen atmosphere. After completion of the reaction, DMF was distilled off completely under reduced pressure to obtain crude product. The crude product was washed several times with hexane and diethyl ether to afford 8 (0.240 g, 80%) as yellow solid.

### Procedure for the Synthesis of 4-(8-bromooctyl)-6,7-dimethoxy-2H-chromen-2-one (9)

To a solution of compound 7 (0.2 g, 1 eq.) in 10 ml of dry acetone, was added K2CO3 (0.302 g, 4 eq.) and Mel (0.308 g, 4 eq.). The above mixture was stirred at room temperature for 6 h. After completion of the reaction as indicated by TLC, the reaction mixture was filtered and the solvent was removed by evaporation at vacuum to get crude products, followed by chromatography to afford 9 (0.165 g, 76%) as yellow solid.

### Procedure for the Synthesis (8-(6,7-dimethoxy-2-oxo-2H-chromen-4-yl)octyl)triphenylphosphonium (10)

To a solution of compound 9 (0.120 g, 1 eq.) in dry DMF (6 ml) was added triphenylphosphine (0.087 g, 1.1 eq.) and the resulting mixture was heated to 120° C. for 12 h under nitrogen atmosphere. After completion of the reaction, DMF was distilled off completely under reduced pressure to obtain the crude product. The crude product was washed several times with hexane and diethyl ether to afford 10 (0.127 g, 72%) as a yellow solid.

### Procedure for the Synthesis of octyltriphenylphosphonium (12)

To a solution of compound 11 (0.2 g, 1 eq.) in dry DMF (6 ml) was added triphenylphosphine (0.298 g, 1.1 eq.) and the resulting mixture was heated to 120° C. for 12 h under nitrogen atmosphere. After completion of the reaction, DMF was distilled off completely under reduced pressure to obtain the crude product. The crude product was washed several times with ethyl acetate and diethyl ether to afford 12 (0.277 g, 71%) as colorless liquid.

All compounds were confirmed by 1H NMR spectroscopy.

### Materials and Methods for Examples 2-4

### Cell Culture:

MDA-MB-231 (a Triple negative breast cancer cell line, ATCC) and MCF-10A cells (normal mammary epithelial cells, ATCC) were grown in Dulbecco's Modified Eagle's medium (DMEM) containing 10% FBS, 1% (v/v) Sodium Pyruvate (100 mM), Sodium bicarbonate (26 mM), L-glutamine (4 mM), penicillin (100 units/ml), and streptomycin (100 μg/ml). Cells were maintained in an incubator at 37° C. in a humidified atmosphere of 5% CO₂ and 95% air.

### Transmission Electron Microscopy (TEM):

Transmission electron microscopy studies were performed on a Tecnai T12 microscope (FEI) at 120 kV, and images were taken using an SIS CCD camera; Samples were negatively stained with ammonium molybdate on 200 or 400 mesh carbon-coated copper grids (Ted Pella, Inc.). Before recording micrographs, the grids were air dried.

### Scanning Electron Microscopy (SEM):

Field emission scanning electron microscopic (FESEM) analysis of a Mito-Esc nanoparticle was performed on a Carl Zeiss SIGMA HD field-emission scanning electron microscope.

### Size and Surface Charge Measurements of Mito-Esc Nanoparticles:

The size (hydrodynamic diameter) and the surface charge (zeta potentials) of a Mito-Esc nanoparticle were measured by photon correlation spectroscopy and the electrophoretic mobility on a Zetasizer 3000HSA (Malvern, UK). The size was measured in deionized water with a sample refractive index of 1.33, a viscosity of 0.88 cP and a temperature of 25° C. The size was measured in triplicate. The zeta potential was measured using the following parameters: viscosity, 0.88 cP; dielectric constant, 78.5, and temperature, 25° C.

### Agarose Gel Electrophoresis Retardation Assay:

Gel electrophoresis was performed with agarose gel (1.5% w/v) in tris-acetate-EDTA buffer (40 mM) with one drop of ethidium bromide added (concentration of EtBr stock solution: 0.625 mg/ml in H₂O), at 100 V for 30 min. The siRNA lipoplexes were prepared by complexing siRNA with Mito-Esc, Mito-isoscopoletin and octyl TPP cation at described P⁺/P⁻ ratios. The samples were incubated for 30 min at room temperature prior to addition into a well. The siRNA bands were visualized under UV illumination at 365 nm.

### Cytotoxicity of Mito-Esc and Mito-Esc/siRNA Complexes:

To evaluate cytotoxicity of the Mito-Esc or Mito-Esc lipoplexes with siMnSOD, a trypan blue dye exclusion assay was used. Briefly, cells were seeded in a 12-well plate at a density of 3×10⁴ cells per well and cultured overnight before transfection. Medium was replaced with 0.5 mL fresh serum free DMEM. siMnSOD (40 nM) was complexed either with lipofectamine-2000 for control or with 2.5 μM Mito-Esc in serum free DMEM medium for 30 min before addition into the plates. The cells were incubated for 48 h, and at the end of the experiments, cells were trypsinized, spun at 800 g for 2 min and resuspended in 1 mL fresh medium. The cell suspension (10 μl) was mixed with an equal amount of trypan blue and counted using an automated cell counter (Countess, Life Technologies).

### Western Blotting:

At the end of the treatments, cell pellets were lysed in a RIPA buffer containing protease inhibitor cocktail, phosphatase inhibitor cocktail-2, 3. Proteins were resolved by SDS-PAGE and blotted onto a nitrocellulose membrane and blocked with 5% bovine serum albumin, washed, and incubated with primary antibodies (1:1000) over night at 4° C. The membranes were then washed and incubated for 1 h with anti-rabbit/mouse IgG horseradish peroxidase linked secondary antibodies (1:5000). ECL reagent (Amersham GE) was applied on the membrane prior to developing with a chemiluminescent system (Bio-Rad).

MnSOD siRNA Transfection:

Cells were cultured in 12-well plates at a density of 3×10⁴ cells per well (containing a glass coverslip the day before use). Briefly, florescent siRNA or siMnSOD (40 nM) was complexed either with lipofectamine-2000, as a positive control or with Mito-Esc (2.5 M) in serum free DMEM medium for 30 min before addition into the plates. After incubation of the cells with siRNA complexes for 6 h, the medium was removed and replaced with 1 mL fresh DMEM medium containing 10% FBS and the cells were further incubated for 24 h.

### Confocal Microscopy Imaging:

Briefly, MDA-MB-231 cells were seeded on a coverslip in 12-well plates at a density of 3×10⁴ cells per well in 1 mL complete DMEM and cultured for 12 h. Fluorescent (Cy-5) siRNA was complexed either with lipofectamine-2000 (positive control) or with Mito-Esc (2.5 μM) or parent esculetin (2.5 μM) or with different cationic lipids in serum free DMEM medium for 30 min before addition into the plates. These lipoplexes were added to the cells and incubated for 6 h. After that, cells were washed twice with PBS and fixed with 4% paraformaldehyde for 15 min. Finally, slides were mounted and the cells were imaged using a confocal microscope. Florescent labeled siRNA with Mito-Esc lipoplexes were prepared as described above and incubated with MDA-MB-231 cells for 24 h. The cells were stained by DAPI to stain the nucleus. The cells were mounted and observed under confocal microscope (Olympus, Tokyo, Japan).

### Example 2: Effects of Mito-Esc on Breast Cancer Cell Viability

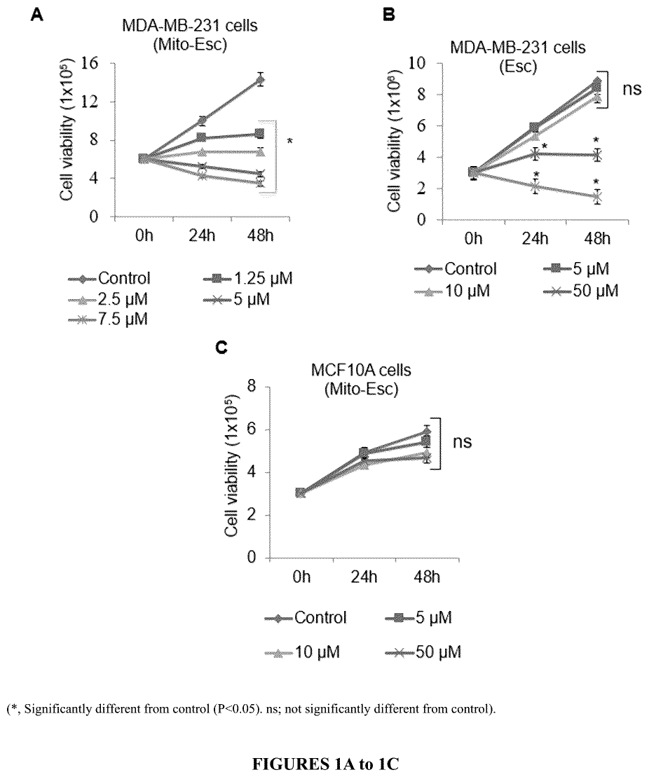

MDA-MB-231 breast cancer cells and MCF-10A (normal mammary epithelial cells) were treated with Mito-Esc and Esc. While Mito-Esc significantly caused a dose-dependent cell death of MDA-MB-231 cells from 1.5-7.5 μM, parent esculetin (Esc) induced cytotoxicity from 50 M (FIGS. 1A and 1B).

Interestingly, Mito-Esc did not show any noticeable toxicity in normal mammary epithelial cells like MCF-10A cells at any of the indicated concentrations (5-50 μM) (FIG. 1C). Thereby showing that Mito-Esc induces anti-proliferative effects preferentially in cancer cells. It was found that Mito-Esc accumulates significantly more in the mitochondrial fraction of MDA-MB-231 breast cancer cells, in comparison to MCF-10A cells. Increased accumulation of Mito-Esc in breast cancer cells induced enhanced mitochondrial superoxide production that in turn depolarized the mitochondrial membrane potential leading to breast cancer cell death.

It is believed that the enhanced uptake of Mito-Esc in cancer cells; including breast cancer cells, is perhaps due to the higher hyperpolarized membrane potential (YIM) possessed by cancer cells, in comparison to normal cells. Delocalized cations (DLCs) pervade easily into the intracellular compartment of cancer cells. Moreover, the hyperpolarized mitochondrial membrane potential of cancer cells (˜−220 mV) as compared to normal cells (˜−140 mV) leads to higher accumulation of DLCs in the mitochondrial fraction. This phenomenon can be of high importance in siRNA therapeutics to maximize the anti-proliferative effect in combination with anti-cancer related siRNAs to preferentially induce cytotoxicity in cancer cells.

Thus, Mito-Esc accumulates more in cancer cells, in comparison to normal cells, thus preferentially causing cancer cell death at significantly lower concentrations.

### Example 3: Effects of Mito-Esc on Viability in Different Cancer Cell Lines

Cytotoxicity of Mito-Esc on different cancer cell lines HeLa (cervical), HepG2 (liver), MCF-7 (ER+ve breast), A549 (lung), DU-145 (prostate) cancer cells and MCF-10A (normal mammary epithelial cells) were determined by treating with Mito-Esc (0.5-100 μM) for 24 h and cell viability was measured by Sulforhodamine B assay.

While Mito-Esc significantly caused a dose-dependent cell death of HeLa (cervical), HepG2 (liver), MCF-7 (ER+ve breast), A549 (lung) cells, as shown in Table 1, it did not show any noticeable toxicity in normal mammary epithelial cells like MCF-10A cells. Thereby showing that Mito-Esc induces anti-proliferative effects preferentially in cancer cells.

TABLE 1

| Cell type | IC50 (μM) |
|---|---|
| Hela | 4.9 |
| HepG2 | 10.0 |
| MCF-7 | 4.8 |
| A549 | 1.8 |
| DU-145 | 15.5 |
| MCF-10A | >100 |

### Example 4: Self-Assembly of Mito-Esc into Nanoparticles

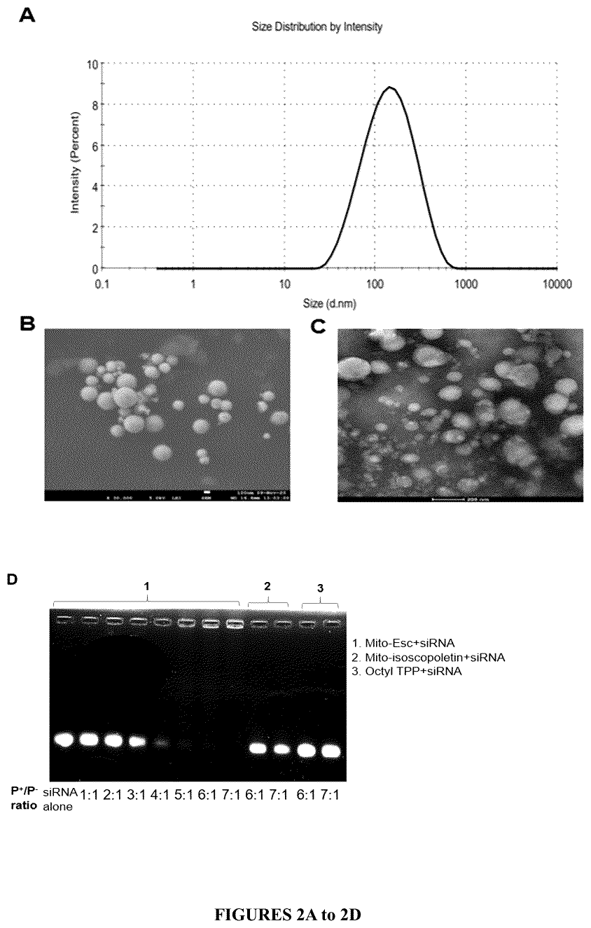

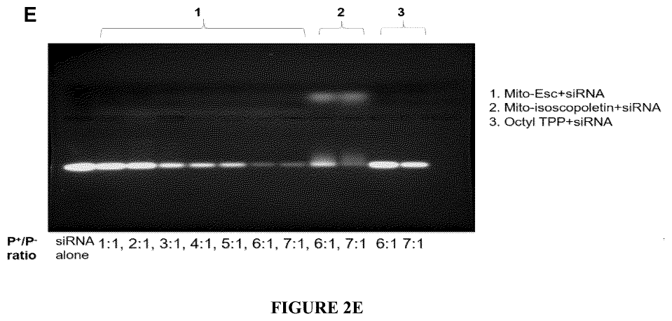

The self-assembling properties of Mito-Esc were explored. The particle size of an aqueous solution (1% EtOH) of Mito-Esc was measured using Dynamic light scattering (DLS). Mito-Esc formed nanosized particles of size 166±30 nm and surface charge of 33±0.4 mV (FIG. 2A). The size and morphology of self-assembled nanoparticles of Mito-Esc was investigated by scanning and by transmission electron microscopy respectively. Mito-Esc formed spherical shaped nanoparticles of <200 nm (FIGS. 2B & 2C). This finding indicates that Mito-Esc can attain self-assembly architecture in aqueous solution.

### Example 5: In-Vivo Orthotopic Tumor Model

Inoculation of MDA-MB-231 cells into mammary fat pad of SCID mice: 6-week-old female SCID mice were used for the experiment. Initially, 10 nM Qtracker labeling solution (Qtracker Cell Labeling Kit, Invitrogen Q25071MP) was prepared by pre-mixing 10 μL each of Component A and Component B in a 1.5 mL microcentrifuge tube and incubated for 5 minutes at room temperature. This mixture was added to 0.2 mL of fresh complete growth medium and vortexed for 30 seconds and added to a 75-cm2 tissue culture flask containing MDA-MB-231 cells and incubated in a 37° C., 5% CO2 incubator overnight. Sub-confluent labeled cells were harvested and counted.

Cells (1×10⁶) were suspended in 0.1 ml of serum free medium. To this 0.1 ml of matrigel was added and gently mixed to get a uniform cell suspension. The SCID mice were anesthetized with ketamine/xylazine cocktail (50 μL/20 g mice) and each mouse was implanted with above mentioned 1×10⁶ Q-Tracker labelled MDA-MB-231 cells into the 4th pair of mammary fat pad orthotopically and sutured the fat pad after the inoculation of cells. The incision site was dressed with povidone-iodine to prevent infection every day until the incision healed. Mice were checked to see development of tumors and treatment began once the tumors reached a volume of ≥300 mm³. Mice were weighed and divided into 4 groups (n=4 per group) and were administered intra peritoneally (i.p) with either esculetin 6 mg/kg·bd·wt) or Mito-Esculetin (3 and 6 mg/kg·bd·wt) for two weeks. On the day of sacrifice, tumor volumes were measured using Vernier calipers. Mice were anaesthetized and sacrificed using cervical dislocation. Tumors were carefully excised, weighed and stored in liquid nitrogen for further histopathological analysis.

### Example 6: Mito-Esc as an Effective siRNA Delivery Vector

### Mito-Esc/siRNA Complexation for Agarose Gel Electrophoresis Retardation Assay:

Mito-Esc nanoparticle solutions were prepared at various concentrations according to the desired final P+:P− charge ratio. Twenty μL solution of the Mito-Esc/siRNA complex was prepared to maintain a constant amount of siRNA in each solution (1 μg in 10 μL), and varying the amount of Mito-Esc according to the P+:P− charge ratio. The prepared mixtures were gently vortexed for 5 min. and incubated for 30 min at room temperature for complex formation. Complexing 1 μg of siRNA with 2, 4, 6, 8, 10, 12, 14 μg of Mito-Esc resulted in 1:1, 2:1, 3:1, 4:1, 5:1, 6:1, 7:1 P+:P− charge ratios respectively.

The Efficiency of Mito-Esc to Bind siRNA:

The efficiency of Mito-Esc to bind siRNA was checked by agarose gel electrophoresis. Also, in order to assess the importance of hydrogen bonding in forming the stable siRNA complexes, Mito-isoscopoletin was synthesized by protecting the dihydroxy group with methyl group. Octyl TPP cation was used as a negative control.

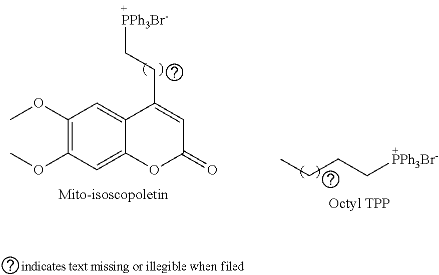

In earlier findings, it was found that only Mito-Esc was able to bind the siRNA from 4:1 to 7:1 P+/P− charge ratios that in turn retarded the siRNA migration (FIG. 2D). In contrast, Mito-isoscopoletin and octyl TPP did not bind the siRNA as evidenced by their inability to retard the siRNA migration even at 7:1 P+/P− charge ratios. These results indicated that the presence of dihydroxy substitution in Mito-Esc facilitates the formation of self-assembled nanoparticles as well as forming stable complexes with siRNA.

However, upon further experiments, we have now found that both Mito-Esc, as well as Mito-isoscopoletin bind the siRNA from P+/P− charge ratios of 4:1 to 7:1 and 6:1 to 7:1, respectively that in turn retarded siRNA migration (FIG. 2E). In contrast, octyl TPP again did not bind the siRNA as evidenced by its inability to retard siRNA migration even at a 7:1 P+/P− charge ratio. These results indicate that H-bonding is not involved in forming the stable siRNA complexes.

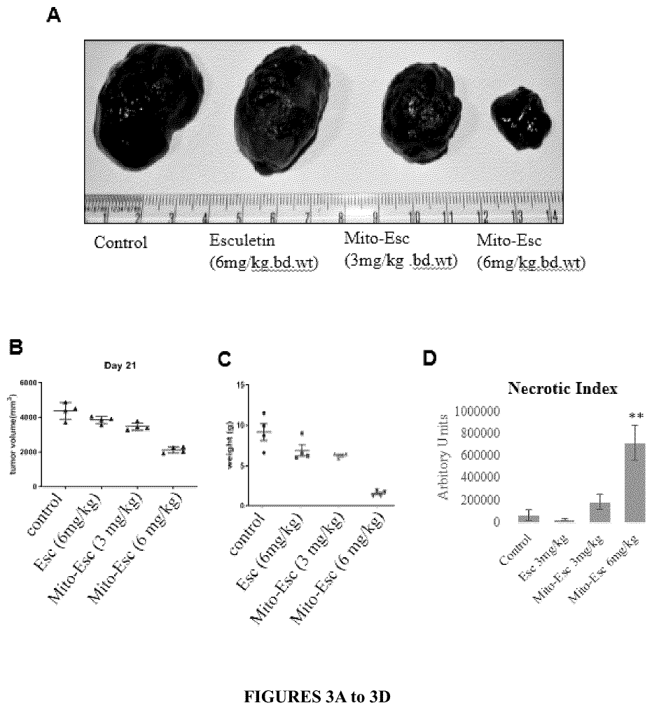

The efficiency of Mito-Esc as a siRNA delivery vector was tested in MDA-MB-231 breast cancer cells. The lipoplex was formed with a custom MnSOD siRNA sequence. It is to be noted that depletion of MnSOD levels in breast cancer cells causes an anti-proliferative effect. MDA-MB-231 cells were treated with the lipoplex for 6 h in an Opti-MEM medium containing reduced serum (˜2%) after which, media was replaced by serum containing medium (10% serum) for another 48 h and measured the cell viability by trypan blue dye exclusion method. In parallel, siMnSOD was also complexed with lipofectamine-2000 (positive control). It was found that Mito-Esc complexed with siMnSOD induced 94% cell death in MDA-MB-231 cells, while Lipofectamine-2000 and siMnSOD complex induced 66% cell death (FIG. 3A).

Further, the gene silencing efficiency of Mito-Esc/siMnSOD complex and lipofectamine-2000/siMnSOD complexes significantly reduced MnSOD protein levels to a similar extent by immunoblotting. In contrast, neither parent esculetin nor octyl TPP cation complexes were able to decrease MnSOD expression (FIG. 3B). These results suggest that Mito-Esc not only preferentially induces breast cancer cell death, but also has got all the structural requirements to form stable complexes with siRNA. Thus, Mito-Esc successfully delivers therapeutic siRNAs, and maximizes the cytotoxic potential in breast cancer cells, proving that Mito-Esc acts as an effective siRNA delivery vector.

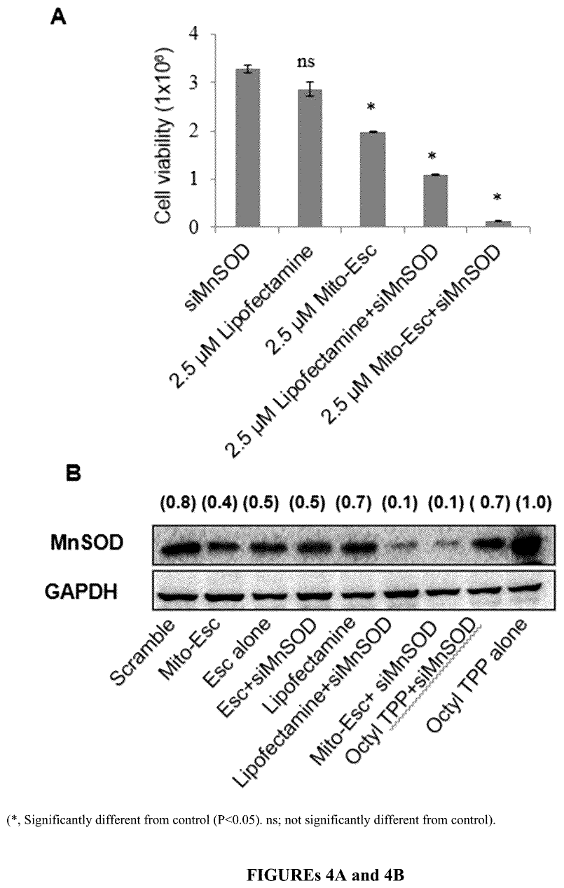

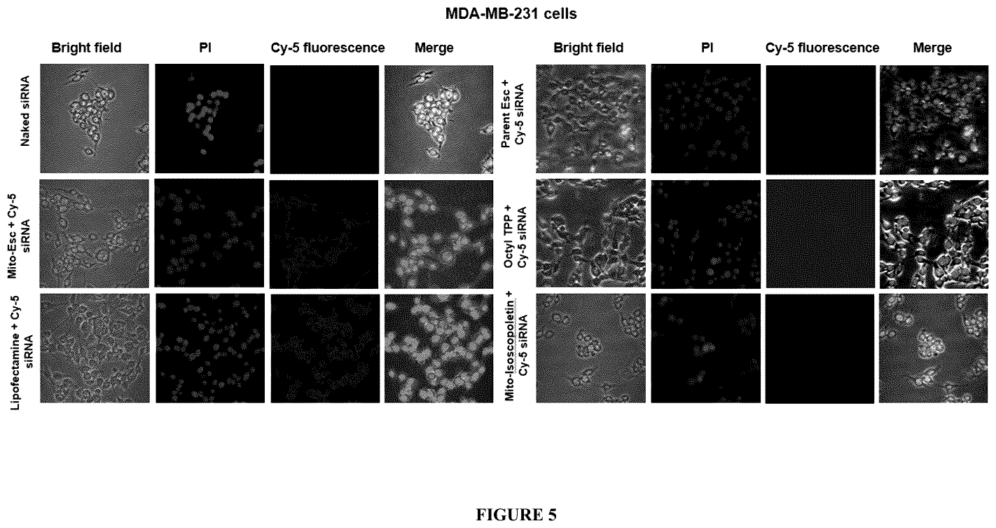

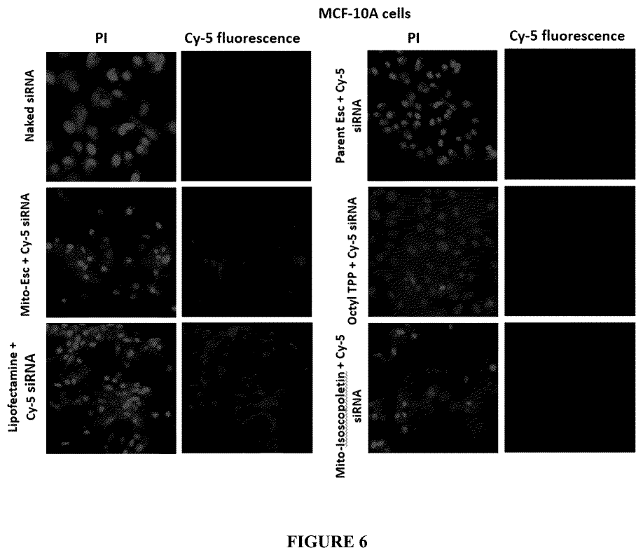

The intracellular delivery of siRNA with Mito-Esc aggregates in MDA-MB-231 and MCF-10A cells was further validated by confocal imaging technique using fluorescently labelled Cy-5 siRNA. In agreement with the results shown in FIG. 3, like Lipofectamine-2000, Mito-Esc significantly caused the intracellular delivery of Cy-5 siRNA (FIG. 4; blue fluorescence). Notably, efficiency of Mi-to-Esc-mediated siRNA delivery was lower than Lipofectamine-2000 in non-cancerous mammary epithelial cell line, MCF-10A, indicating a potential cancer cell-selective siRNA delivery by Mito-Esc (FIG. 5). In contrast, parent esculetin, Mito-isoscopaletin and octyl TPP failed to deliver the Cy-5 siRNA intracellularly, possibly due to their inability to form stable complexes with siRNA. These results substantiate that Mito-Esc acts as an effective siRNA delivery vector.

1. A complex of a 6,7-dihydroxy coumarin phosphonium amphiphile and a negatively charged agent.

2. The complex as claimed in claim 1, wherein the 6,7-dihydroxy coumarin phosphonium amphiphile is selected from: 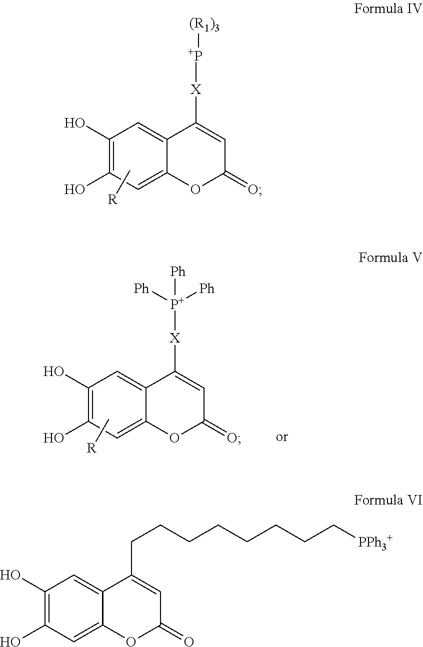
wherein,
R is hydrogen, a substituted alkyl, a substituted aryl or a substituted heteroatom;
R₁ is an aryl, a cycloalkyl or a heteroaryl;
X is a C₁ to C₃₀ carbon chain including one or more double or triple bonds, unsubstituted or substituted with alkyl, alkenyl or alkynyl side chains, or 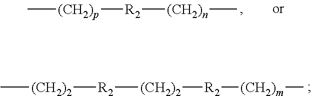
wherein
p is 2 or 3, n is an integer from 3 to 6, and m is an integer from 2 to 4; and
R₂ is O, NH or S.

3. The complex as claimed in claim 1, wherein the complex is
(i) a compound of Formula I: 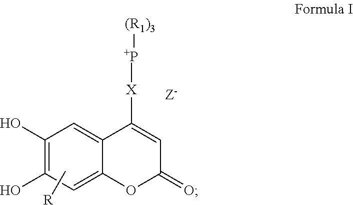
wherein,
Z is a negatively charged agent;
R is hydrogen, a substituted alkyl, a substituted aryl or a substituted heteroatom;
R₁ is an aryl, a cycloalkyl or a heteroaryl;
X is a C₁ to C₃₀ carbon chain including one or more double or triple bonds, unsubstituted or substituted with alkyl, alkenyl or alkynyl side chains, or 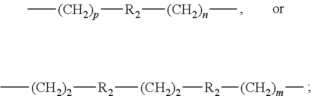
wherein,
p is 2 or 3, n is an integer from 3 to 6, and m is an integer from 2 to 4; and
R₂ is O, NH or S,
(ii) a compound of Formula II: 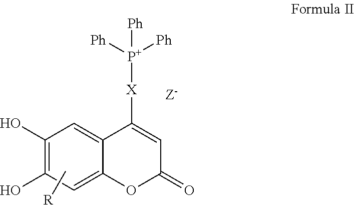
wherein,
X is a C₁ to C₃₀ carbon chain;
Z is a negatively charged agent; and
R is hydrogen, one or more substituted alkyl, one or more substituted aryl or one or more substituted heteroatom, or
(iii) a compound of Formula III: 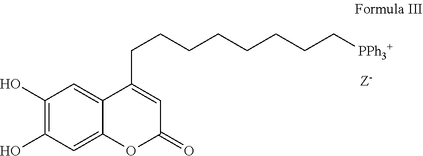
wherein,
Z is a negatively charged agent.

4. (canceled)

5. (canceled)

6. The complex as claimed in claim 1, wherein the negatively charged agent is selected from a therapeutic agent, a diagnostic agent or a nucleic acid.

7. The complex as claimed in claim 6, wherein the nucleic acid is selected from small interfering RNAs (siRNAs) and messenger RNA (mRNA).

8. The complex as claimed in claim 1, wherein the complex is in the form of nanoparticle.

9. A method of treating, ameliorating or diagnosing cancer, wherein said method comprises administering an effective amount of a complex as claimed in claim 1.

10. (canceled)

11. A pharmaceutical composition comprising a complex as claimed in claim 1 and a pharmaceutically acceptable carrier or excipient.

12. A pharmaceutical composition comprising a complex of claim 16 with a pharmaceutically acceptable carrier or excipient.

13. (canceled)

14. A method of intracellular delivery of a negatively charged agent, said method comprising administering an effective amount of a complex as claimed in claim 1.

15. A method of intracellular delivery of a negatively charged agent, said method comprising administering an effective amount of a pharmaceutical composition as claimed in claim 11.

16. A complex of Mito-Esc and siRNA, wherein Mito-Esc is represented by a compound of Formula VI:

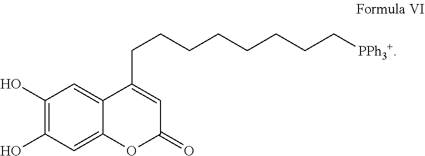

17. A nanoparticle comprising the complex of claim 1.

18. (canceled)

19. (canceled)

20. (canceled)

21. (canceled)

22. The nanoparticle as claimed in claim 17, wherein the negatively charged agent is a therapeutic agent, a diagnostic agent or a nucleic acid

23. The nanoparticle as claimed in claim 22, wherein the nucleic acid is selected from small interfering RNAs (siRNAs) and messenger RNA (mRNA).

24. (canceled)

25. The nanoparticle as claimed in claim 17, wherein the nanoparticle has a size of 100 to 200 nm.

26. (canceled)

27. (canceled)

28. (canceled)

29. (canceled)

30. A pharmaceutical composition comprising a nanoparticle of claim 34 with a pharmaceutically acceptable carrier or excipient.

31. (canceled)

32. A method of intracellular delivery of a negatively charged agent, said method comprising administering an effective amount of a nanoparticle as claimed in claim 17.

33. A method of intracellular delivery of a negatively charged agent, wherein said method comprising administering an effective amount of a pharmaceutical composition as claimed in claim 30.

34. A nanoparticle comprising the complex of claim 16.

35. A compound of
(i) Formula I: 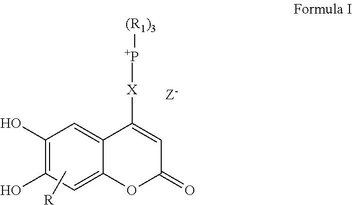
wherein,
Z is a negatively charged agent or a negatively charged counterion;
R is hydrogen, a substituted alkyl, a substituted aryl or a substituted heteroatom;
R₁ is an aryl, a cycloalkyl or a heteroaryl;
X is a C₁ to C₃₀ carbon chain including one or more double or triple bonds, unsubstituted or substituted with alkyl, alkenyl or alkynyl side chains, or 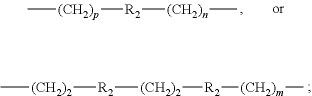
wherein,
p is 2 or 3, n is an integer from 3 to 6, and m is an integer from 2 to 4; and
R₂ is O, NH or S,
(ii) Formula II: 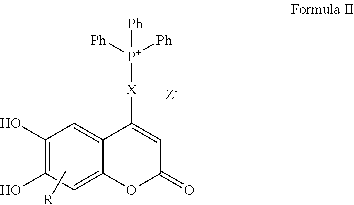
wherein,
X is a C₁ to C₃₀ carbon chain;
Z is a negatively charged agent or a negatively charged counterion; and
R is hydrogen, one or more substituted alkyl, one or more substituted aryl or one or more substituted heteroatom, or
(iii) Formula III: 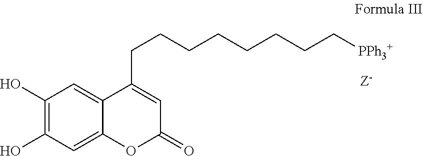
wherein,
Z is a negatively charged agent or a negatively charged counterion.

36. (canceled)

37. (canceled)

38. The compound as claimed in claim 35, wherein the negatively charged agent is selected from a therapeutic agent, a diagnostic agent or a nucleic acid.

39. The compound as claimed in claim 38, wherein nucleic acid is selected from small interfering RNAs (siRNAs) and messenger RNA (mRNA).

40. The compound as claimed claim 35, wherein the negatively charged counterion is selected from mesylate, tosylate, citrate, tartrate, malate, acetate and trifluoroacetate.

41. A method of treating, ameliorating or diagnosing cancer, wherein said method comprises administering an effective amount of a compound as claimed in claim 35.

42. (canceled)

43. A pharmaceutical composition comprising a compound as claimed in claim 35 and a pharmaceutically acceptable carrier or excipient.

44. A pharmaceutical composition comprising a compound of Formula III: 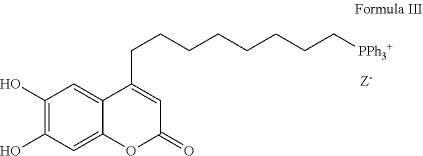
wherein, Z is siRNA;
with a pharmaceutically acceptable carrier or excipient.

45. (canceled)

46. A method of intracellular delivery of a negatively charged agent, said method comprising administering an effective amount of a compound as claimed in claim 35.

47. (canceled)

48. A compound of Formula III: 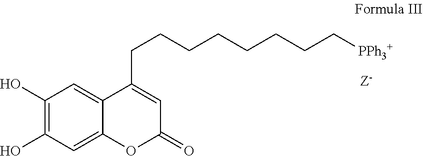
wherein,
Z is siRNA.

49. (canceled)

50. (canceled)

51. (canceled)

52. (canceled)

53. (canceled)

54. (canceled)

55. (canceled)
